(19) United States
(12) Patent Application Publication
(10) Pub. No.: US 2024/0293645 A1
(43) Pub. Date: Sep. 5, 2024

(54) MULTI-LUMEN CENTRAL LINE

(71) Applicant: Harold Nathanial Claver, Ardmore, OK (US)

(72) Inventor: Harold Nathanial Claver, Ardmore, OK (US)

(21) Appl. No.: 18/578,538

(22) PCT Filed: Jun. 16, 2023
(86) PCT No.: PCT/US2023/068605

§ 371 Date: Jan. 11, 2024

Related U.S. Application Data
(60) Provisional application No. 63/353,015, filed on Jun. 16, 2022.

(51) Int. Cl. A61M 25/06; A61M 25/00

(52) U.S. Cl. CPC A61M 25/0606; A61M 25/0097; A61M 25/0693

(57) ABSTRACT

An apparatus for introducing medications and fluids into a patient configured for using a catheter over needle technique to place a central line into the bloodstream of a patient, the apparatus comprising a multi-lumen line extending between a proximal end of the catheter and a distal end, the multi-lumen line comprising a central lumen and at least one non-central lumen, the central lumen having a size larger than that of the at least one non-central lumen, whereby the central lumen and at least one non-central lumen are capable of being utilized to draw blood or deliver fluids or medication and a catheter junction hub at the proximal end, the catheter junction hub separating the multi-lumen line, the catheter junction hub comprising a port opening to the central lumen, whereby the apparatus is capable of utilizing a syringe or flashback chamber to place the multi-lumen line into the bloodstream of the patient.

### RELATED APPLICATIONS

This application is being filed under U.S. national stage under 35 USC 371 of PCT Application No. PCT/US2023/068605 filed on Jun. 16, 2023 and entitled “Multi-Lume Central Line,” which claims priority from U.S. Provisional Application No. 63/353,015 filed on Jun. 16, 2022 and entitled “Multi-Lumen Central Line,” the entire contents of which are hereby fully incorporated herein.

### BACKGROUND OF THE INVENTION

The invention relates to an improved method and system for accessing the vasculature of a patient. The invention further relates to a technique for delivering fluids and medications to patients.

### 1. Field of the Invention

The present invention relates to the field of placing a central line in the vasculature of a patient using a catheter over needle technique. The present invention further relates minimally invasive IV placement. The present invention even further relates to reducing the time and discomfort that a patient experiences when receiving a central line.

### 2. Description of Related Art

Traditional placement of a central line using the Seldinger technique is time consuming and unpleasant for the patient. While the Seldinger technique is widely used, it involves a great deal of steps and parts. This has two primary disadvantages. First, the longer sequencing and multiple parts involved increases the potential for patient trauma. Second, the numerous steps involved with multiple parts entering and exiting the patient's blood stream during the procedure increases the risk of introducing contaminants into the patient, who often is immunocompromised and at risk for further infection. The Seldinger technique has the additional disadvantage of needing a high degree of sterility due to the numerous steps and multiple parts. See FIG. 4a-c for a depiction of the needle over catheter central line. Thus, there is a need to reduce the number of steps and parts required to place a central line.

There remains a solution for a method and system needed to improve upon the traditional placement of a central line.

### SUMMARY OF THE INVENTION

These and other objects were met with the present invention. The present invention provides an apparatus for introducing medications and fluids into a patient configured for using a catheter over needle technique to place a central line into the bloodstream of a patient, the apparatus comprising a multi-lumen line extending between a proximal end of the catheter and a distal end, the multi-lumen line comprising a central lumen and at least one non-central lumen, the central lumen having a size larger than that of the at least one non-central lumen, whereby the central lumen and at least one non-central lumen are capable of being utilized to draw blood or deliver fluids or medication and a catheter junction hub at the proximal end, the catheter junction hub separating the multi-lumen line, the catheter junction hub comprising a port opening to the central lumen, whereby the apparatus is capable of utilizing a syringe or flashback chamber to place the multi-lumen line into the bloodstream of the patient; the apparatus further comprising a sealing element applied to the at least one non-central lumen; the central lumen having a volumetric value in the range of 16 g through 18 g, the at least one non-central lumen having a volumetric value in the range of 18 g through 22 g; the central lumen having a volumetric value in the range of 12 g through 24 g, the at least one non-central lumen having a volumetric value in the range of 12 g through 24 g; the central lumen having a distal end and a proximal end, the central lumen being tapered at the distal end of the multi-lumen line; the multi-lumen having a length of between 4 cm and 20 cm; the at least one non-central lumen extending beyond the multi-lumen, journaling therethrough the catheter junction hub towards the proximal end into an independent lumen, whereas the sealing element seals the independent lumen; whereas the sealing element is a clamping device; the catheter junction hub having at least one member extending outwardly, the at least one member configured for stabilization upon a patient's skin; the catheter junction hub having at least one upper member and at least one lower member; the catheter junction hub having at least one aperture therethrough capable of accessing patient's skin surface; the catheter junction hub having a port being capable of mating with a flashback chamber, a syringe, and a luer connection.

In another embodiment of the invention, a method is disclosed comprising delivering a catheter over needle technique to place a central line is disclosed, the method comprising the following steps, coupling a multi-lumen line with a syringe or a flashback chamber having a needle, the multi-lumen having a central lumen and a non-central lumen, the central lumen having a different size than the non-central lumen, inserting the needle through a luer connecter on the hub into the multi-lumen line into the patient's vasculature, enabling the multi-lumen to be placed over the needle through the vasculature and into the patient's bloodstream; and withdrawing the needle and securing the multi-lumen line for use, whereby the step of coupling a multi-lumen line with a syringe or a flashback chamber having a needle further utilizes a catheter junction hub that allows the needle to access to the central lumen; the method further comprising the step of controlling the access to the central lumen with a luer lock or other luer connector placed on the catheter junction hub, controlling access to the central lumen; the method further comprising the step of enabling the non-central lumen to be selectively sealed or maintain a static pressure during use; the method further comprising the step of drawing blood from the patient through the central lumen, at least one non central lumen, or any independent lumen; the method further comprising the step of delivering medication or fluids via the central lumen, the at least one non-central lumen, or any independent lumen; the method further comprising the step of separating the multi-lumen as it enters the catheter junction hub into independent lumen that emerge outside of the catheter junction hub and sealing the independent lumen with a clamping device.

### BRIEF DESCRIPTION OF THE DRAWINGS

A more complete understanding of the present invention may be derived by referring to the detailed description and claims when considered in connection with the Figures, where like reference numbers refer to similar elements throughout the Figures:

FIG. 1a depicts a top plan view of the invention according to embodiments;

FIG. 1b depicts a cross section view of 1a at the multi-lumen line;

FIG. 2 depicts a syringe for use with the inventive system;

FIG. 3 illustrates a flashback chamber for use with the inventive system,

FIGS. 4a, 4b, and 4c, 4d, and 4e depict a front view of a central catheter utilizing the catheter over needle technique in a sequence of placement into a patient.

FIG. 5 depicts a syringe in use with the inventive system in FIGS. 4a-e.

FIG. 6 illustrates a luer lock connection with the inventive system in FIGS. 4a-e.

### DETAILED DESCRIPTION OF THE INVENTION

The present invention solves a number of issues with placing a central line into a patient. The present invention provides a solution that is as easily and quickly deployed as a peripheral IV. The present invention may reduce the need to over-sterilize the components of the system. The invention further provides a more comfortable procedure for the patient. The invention provides a shorter central line enabling an easier experience with provider placement of the central line. The invention even further provides a shorter catheter line that utilizes a catheter over needle technique superior to that in the current arts.

According to the illustrations provided, a method and system of introducing intravenous fluids and medication to a patient are disclosed.

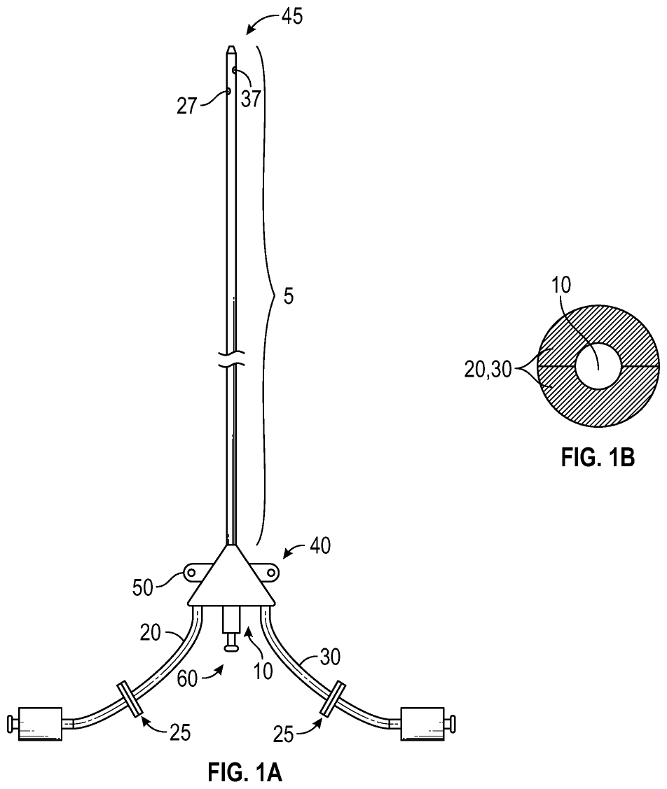

In an embodiment of the inventive system as seen in FIG. 1a, the system uses the catheter over needle technique to place a central line into a patient's bloodstream. Though a traditional central line is approximately 20 cm long, the inventive system may comprise a lesser length, thereby enabling the needle over catheter technique to be used through the central lumen. In an embodiment of the inventive system, the lumens may be approximately 8 cm, or in a range of between 8 cm and 12 cm. In other embodiments, the lumens may be within a range of 2 cm to 18 cm. In yet another embodiment, a first, second and third lumen may have different lengths within the range of 2 cm to 18 cm.

The inventive system may comprise a triple lumen 5 comprising at least two volumetric values. In one embodiment of the invention, 3 separate lumen may comprise a central lumen 10 having a volumetric value of 18 g, and a second 20 and third 30 lumens may have a volumetric value of 22 g each. In other embodiments of the invention, the ranges of the volumetric value may be 12 g-24 g for the central line, and 12 g-24 g for the second and third lumen. Looking further at FIG. 1b, the central lumen 10 is shown to be internal of second 20 and third 30 lumen. Other configurations and arrangements of central, second and third lumen are well within the scope of the invention, including those where the central lumen is partially internal or external of the second and third lumen. The triple lumen may comprise a catheter junction hub 40, where the second 20 and third 30 lumen are separated from a port 60 of the catheter junction hub 40. After the second 20 and third 30 lumen emerge from the catheter junction 40, clamps 25 may be applied to ensure a static pressure is maintained. In other embodiments, the second 20 and third 30 lumen may have other configurations outside of the catheter junction than those shown in FIG. 1, well within the scope of the invention.

In still another embodiment of the invention, a multi-lumen may comprise a central lumen 10, and at least one non-central lumen. A multi-lumen catheter may extend between a patient's bloodstream and a catheter junction hub 40, separating catheter streams to both deliver medication and fluids as well as draw blood through the central lumen 10 or any of the at least one non-central lumen. In another embodiment of the multi-lumen, the central lumen 10 may will terminate more distal than that of the at least one non-central lumen, enabling the system to self-dilate during insertion. In another embodiment of the multi-lumen, the distal ends of the any of the lumen may taper, enabling the system to self-dilate during insertion. As depicted, the central lumen 10 is shown in FIG. 1b to have a slightly larger gauge of line than that of the non-central lumen. The central lumen 10 may have a distal end 45 at the top of FIG. 1a and a catheter junction end 60 at the bottom of FIG. 1a. At the distal end, the catheter may comprise terminal points 27 and 37 for the non-central lumen 20 and 30, the terminal points 27 and 37 positioned along the portion of the multi-lumen that is configured to be placed within the patient's bloodstream. As such, terminal points 27 and 37 may communicate the contents of the non-central lumen with the patient bloodstream, and further to drawn blood from the patient into one of the non-central lumen 20, 30. The distal end 45 of the catheter may be tapered in various embodiments of the invention. FIG. 1 shows an embodiment of the tapered multi-lumen between the terminal points 27, 37 and the distal end 45. In other embodiments, the tapered multi-lumen may take place in increments or smoothed transitions through the terminal points 27, 37. Though an example of the tapered end is shown in FIG. 1a, it should not be understood as a limitation as to the scope of how the triple lumen is constructed, as it may have other geometric configurations.

In another embodiment of the invention, the multi-lumen may extend proximally past the catheter junction hub, separating into independent lines inside or outside the catheter junction hub. In yet another embodiment not depicted, the central lumen may be separated from the non-central lumen lines that remain consolidated to source inputs of medication or other beneficial additives.

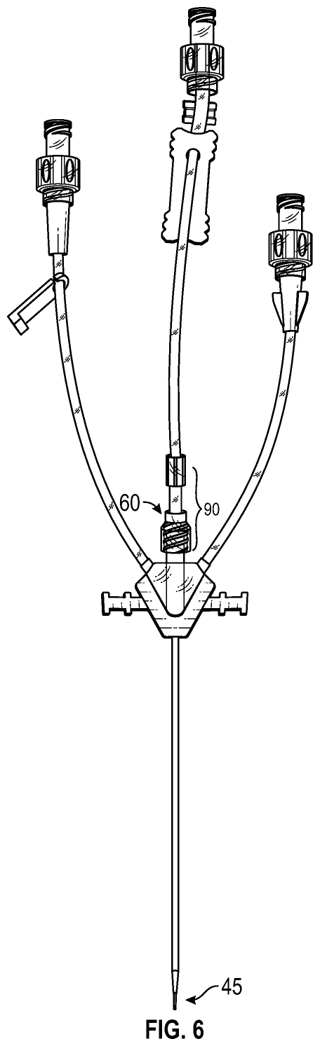

In another embodiment of the invention seen in FIG. 6, a lumen luer connection 90 may directly or indirectly communicate with the catheter junction hub 40. Lumen luer connection 90 may access the central lumen 10 through a port 60 through the catheter junction hub 40 as shown. In other embodiments, the lumen luer connection 90 may access the central lumen 10 through other connections well known in the arts. In still another alternative embodiment, a non luer connecter interface with the catheter junction hub and connect with any needleless connector to deliver regulated inputs to a patient. Such connections may enable the user to deliver a catheter over needle technique through the inventive system in FIG. 1a.

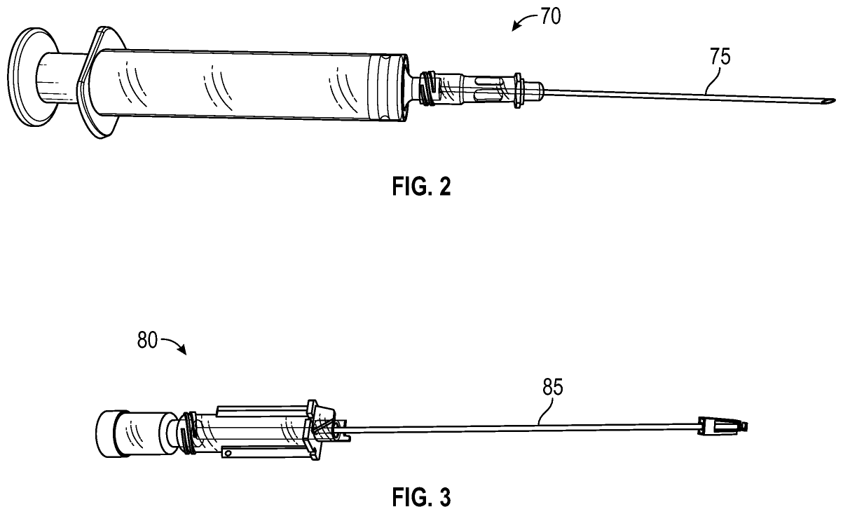

In another embodiment of the invention, the catheter may be inserted with a syringe and needle 70 shown in FIG. 2 to aspirate blood from a patient via to ensure proper placement. The needle 75 of the syringe may be inserted through the central lumen 10, the catheter may be tapered towards a distal end 45 of the catheter at the distal end 45. Thus, the catheter may be fed over the needle 75 of the syringe 70.

In yet another embodiment of the invention, the catheter may be utilized through its port 60 via a flashback chamber 80 as illustrated in FIG. 3. The needle 85 of the flashback chamber 80 may be inserted through the port 60, therethrough the catheter 5 which may be tapered as it nears the distal end 45 of the catheter. Thus, the catheter may be fed over the needle 85 of the syringe 70. In another embodiment of the invention, a process for delivering a catheter over needle technique to place a central line is disclosed. Depending upon the patient's needs and the medical objectives, an inventive multi-lumen such as one illustrated in FIG. 1a may be coupled with a syringe (FIG. 2) or a flashback chamber (FIG. 3). The syringe or flashback chamber may connect to a catheter junction hub on the proximal side of the hub. The needle of the syringe or flashback chamber may then be inserted into the patient's vasculature, enabling the catheter 5 of the inventive system to be placed through the vasculature and into the patient's bloodstream. The needle may be withdrawn, and the inventive system secured for use.

A luer lock or other luer connector may be placed on the central luer connector of the catheter junction hub, controlling access to the central lumen. The multi-lumen may comprise a central lumen and at least one non-central lumen extending between the distal end of the catheter and the catheter junction hub. In alternative embodiments, the catheter 5 may be consolidated or separate lumen between the patient's bloodstream and the catheter junction hub. The inventive method may further include separating the multi-lumen as it enters the catheter junction hub into one or more independent lumens, enabling the independent lumen to be selectively sealed or of a static pressure; Draw blood from the patient through the central lumen, at least one non central lumen, or any independent lumen; Deliver medication or fluids via the central lumen, the at least one non-central lumen, or any independent lumen.

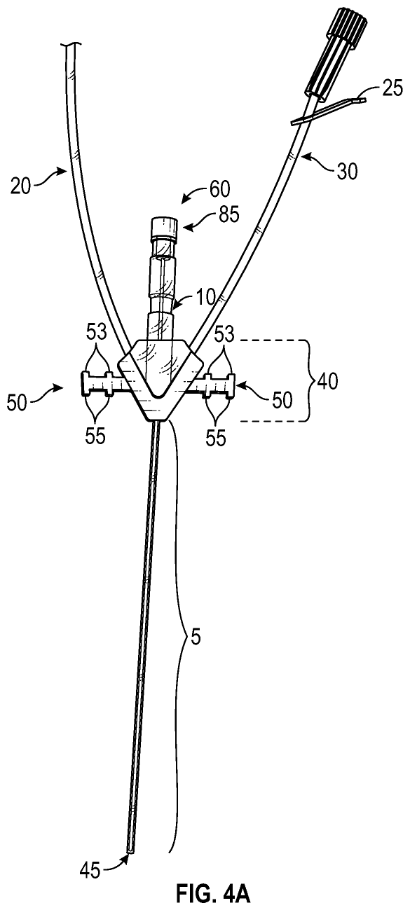

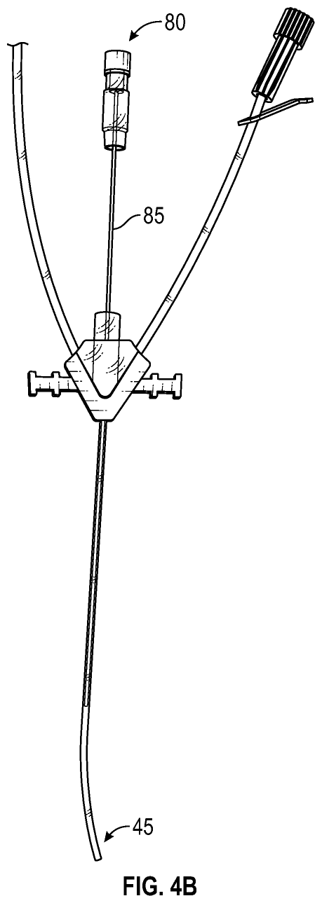

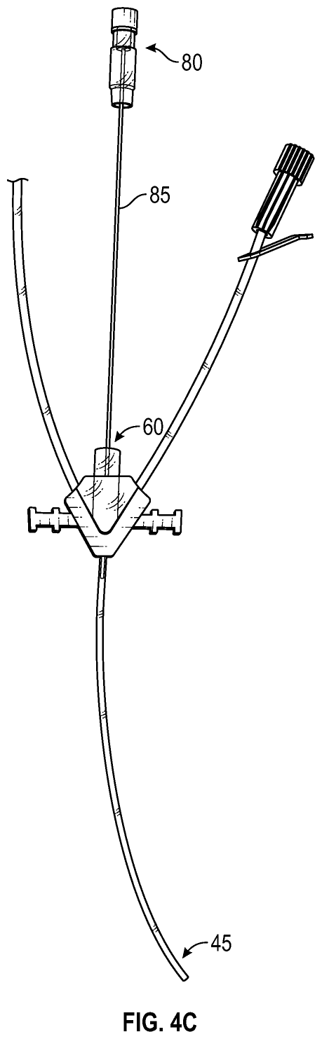

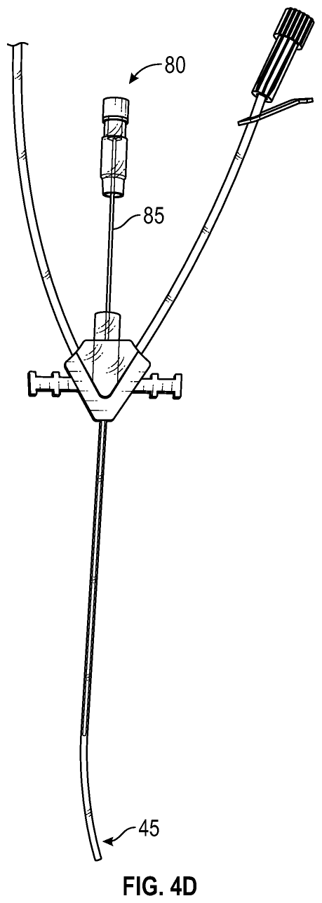

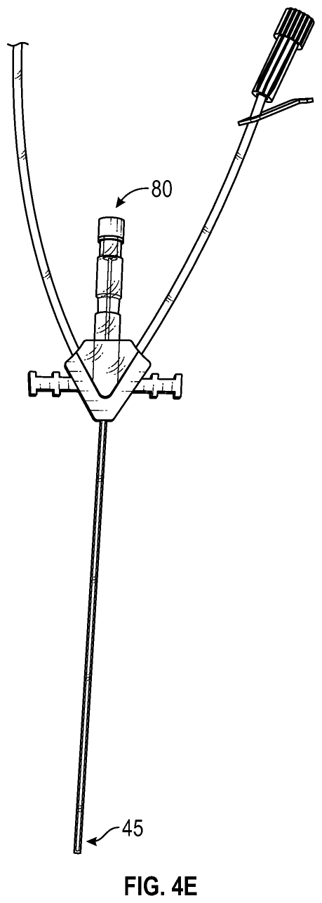

In another embodiment of the invention shown in FIGS. 4a-4e, a system uses the catheter over needle technique to place a central line into a patient's bloodstream. Though a traditional central line is approximately 20 cm long, the inventive system may comprise a lesser length, thereby enabling the needle over catheter technique to be used through the central lumen. In an embodiment of the inventive system, the lumens may be approximately 6 cm, or in a range of between 5 cm and 10 cm. In other embodiments, the lumens may be within a range of 2 cm to 18 cm. In yet another embodiment, a first, second and third lumen may have different lengths within the range of 2 cm to 18 cm.

Looking further at FIG. 4a, the inventive system may comprise a triple lumen 5 comprising at least two volumetric values. In one embodiment of the invention shown in FIG. 1b, 3 separate lumen may comprise a central lumen 10 having a volumetric value of 16 g-18 g, and a second 20 and third 30 lumens may have a volumetric value of 18 g-20 g each. In other embodiments of the invention, the ranges of the volumetric value may be 12 g-24 g for the central line, and 12 g-24 g for the second and third lumen. Looking further at FIG. 1b, the central lumen 10 is shown to be internal of second 20 and third 30 lumen. Other configurations and arrangements of central, second and third lumen are well within the scope of the invention, including those where the central lumen is partially internal or external of the second and third lumen. Looking more closely at FIGS. 4a-4e, the inventive system is shown in use during insertion in the patient. The triple lumen may comprise a catheter junction hub 40, where the second 20 and third 30 lumen are separated from a port 60 of the catheter junction hub 40. Catheter junction hub 40 may be configured in various geometrically diverse structures according to embodiments of the invention. A catheter junction hub may in the present embodiment be understood as a singular union for merging the triple lumen 5 with the input lines, as well as establishing a point of contact with the patient. In other embodiments not shown, catheter junction hub 40 may have disparate parts and functions that are not within a singular union. As shown in FIGS. 4a-e, hub 40 may comprise at least one member 50, and as shown a first member 50 and a second member 50 extending from hub 40. Members 50 as shown may include at least one member elements, and as shown upper member elements 53 and lower member elements 55. Upper member elements 53 and lower member elements 55 may be utilized for attaching the catheter junction hub 40 to a patient's skin through various means, including medical tape, wrap, suture staple and/or other skin attaching means known in the medical arts. Though members 50 are shown as extending in an approximate perpendicular relationship to triple lumen 5, it is well within the embodiments of the invention for member or members 50 to extend in other directions from the hub 40. Further, member or members 50 may extend toward or away from the patient, not only laterally of the hub 40. In yet other embodiments, hub 40 may be understood to extend in a range of 360 degrees from the x, y and z axes established and referenced from the triple lumen 5. After the second 20 and third 30 lumen emerge from the catheter junction 40, clamps 25 may be applied to ensure a static pressure is maintained. In other embodiments, the second 20 and third 30 lumen may have other configurations outside of the catheter junction than those shown in FIG. 1, well within the scope of the invention.

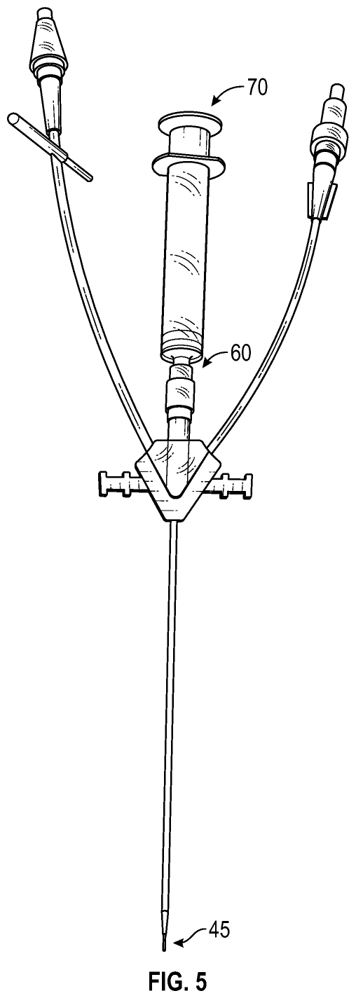

The inventive system may be utilized through its port 60 via a flashback chamber 80 as shown in the sequence illustrated from FIG. 4b-e. The needle 85 of the flashback chamber 80 may be inserted through the port 60, therethrough the catheter 5 which may be tapered as it nears the distal end 45 of the catheter. Looking at another interaction within the process of using the inventive system shown in FIG. 5, the catheter 5 and specifically the central lumen 5 as accessed via the port 60 may be fed over the needle 85 of the syringe 70, allowing the medical professional to introduce the proscribed input isolated through the central lumen 10.

As seen in FIG. 6, the inventive system may interact with a luer lock connection 90, which may directly or indirectly communicate with the catheter junction hub 40. Lumen luer connection 90 may access the central lumen 10 through a port 60 through the catheter junction hub 40 as shown. Port may simply represent an extension of the central lumen as it emerges from the hub 40 in an embodiment of the invention or may include other portions and materials extending between the central lumen entering into the hub 40 and emerging through the port in other embodiments of the invention. In other embodiments, the lumen luer connection 90 may access the central lumen 10 through other connections well known in the arts. In still another alternative embodiment, a non luer connecter interface with the catheter junction hub and connect with any needleless connector to deliver regulated inputs to a patient. Such connections may enable the user to deliver a catheter over needle technique through the inventive system in FIGS. 4a-e, 5 and 6.

In still another embodiment of the invention, a multi-lumen may comprise a central lumen 10, and at least one non-central lumen. A multi-lumen catheter may extend between a patient's bloodstream and a catheter junction hub 40, separating catheter streams to both deliver medication and fluids as well as draw blood through the central lumen 10 or any of the at least one non-central lumen. In another embodiment of the multi-lumen, the central lumen 10 may comprise a different size than that of the at least one non-central lumen, enabling the system to self-dilate during insertion. In another embodiment of the multi-lumen, the distal ends of the any of the lumen may taper, enabling the system to self-dilate during insertion. As depicted, the central lumen 10 is shown in FIG. 1b to have a slightly larger gauge of line than that of the non-central lumen. The central lumen 10 may have a distal end 45 at the top of FIG. 1 and a catheter junction end at the bottom of FIG. 1b. At the distal end, the catheter may comprise terminal points 27 and 37 for the non-central lumen 20 and 30, the terminal points 27 and 37 positioned along the portion of the multi-lumen that is configured to be placed within the patient's bloodstream. As such, terminal points 27 and 37 may communicate the contents of the non-central lumen with the patient bloodstream, and further to drawn blood from the patient into one of the non-central lumen 20, 30. The distal end 45 of the catheter may be tapered in various embodiments of the invention. FIG. 1 shows an embodiment of the tapered multi-lumen between the terminal points 27, 37 and the distal end 45. In other embodiments, the tapered multi-lumen may take place in increments or smoothed transitions through the terminal points 25, 35. Though an example of the tapered end is shown in FIGS. 4a-c, it should not be understood as a limitation and may have other geometric configurations not illustrated herein.

Other variations and specialized circumstances may be implemented with this.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. An apparatus for introducing medications and fluids into a patient configured for using a catheter over needle technique to place a central line into the bloodstream of a patient, the apparatus comprising:
a multi-lumen line extending between a proximal end of the catheter and a distal end, the multi-lumen line comprising a central lumen and at least one non-central lumen, the central lumen having a size larger than that of the at least one non-central lumen, whereby the central lumen and at least one non-central lumen are capable of being utilized to draw blood or deliver fluids or medication; and
a catheter junction hub at the proximal end, the catheter junction hub separating the multi-lumen line, the catheter junction hub comprising a port opening to the central lumen, whereby the apparatus is capable of utilizing a syringe or flashback chamber to place the multi-lumen line into the bloodstream of the patient.

2. The apparatus in claim 1 further comprising a sealing element applied to the at least one non-central lumen.

3. The apparatus in claim 1, the central lumen having a volumetric value in the range of 16 g through 18 g, the at least one non-central lumen having a volumetric value in the range of 18 g through 22 g.

4. The apparatus in claim 1, the central lumen having a volumetric value in the range of 12 g through 24 g, the at least one non-central lumen having a volumetric value in the range of 12 g through 24 g.

5. The apparatus in claim 1, the central lumen having a distal end and a proximal end, the central lumen being tapered at the distal end of the multi-lumen line.

6. The apparatus in claim 1, the multi-lumen having a length of between 4 cm and 20 cm.

7. The apparatus in claim 5, the at least one non-central lumen extending beyond the multi-lumen, journaling therethrough the catheter junction hub towards the proximal end into an independent lumen, whereas the sealing element seals the independent lumen.

8. The apparatus in claim 2, whereas the sealing element is a clamping device.

9. The apparatus in claim 1, the catheter junction hub having at least one member extending outwardly, the at least one member configured for stabilization upon a patient's skin.

10. The catheter junction hub in claim 9 having at least one upper member and at least one lower member.

11. The catheter junction hub in claim 1 having at least one aperture therethrough capable of accessing patient's skin surface.

12. The catheter junction hub in claim 1, the port being capable of mating with a flashback chamber, a syringe, and a luer connection.

13. A method for delivering a catheter over needle technique to place a central line is disclosed, the method comprising the following steps:
Coupling a multi-lumen line with a syringe or a flashback chamber having a needle, the multi-lumen having a central lumen and a non-central lumen, the central lumen having a different size than the non-central lumen;
Inserting the needle through a luer connecter on the hub into the multi-lumen line into the patient's vasculature;
Enabling the multi-lumen to be placed over the needle through the vasculature and into the patient's bloodstream; and
Withdrawing the needle and securing the multi-lumen line for use.

14. The method of claim 9, whereby the step of coupling a multi-lumen line with a syringe or a flashback chamber having a needle further utilizes a catheter junction hub that allows the needle to access to the central lumen.

15. The method of claim 9 further comprising the step of:
Controlling the access to the central lumen with a luer connector placed on the catheter junction hub, controlling access to the central lumen.

16. The method of claim 9 further comprising the step of:
Enabling the non-central lumen to be selectively sealed or maintain a static pressure during use.

17. The method of claim 9 further comprising the step of:
Drawing blood from the patient through the central lumen, at least one non central lumen, or any independent lumen.

18. The method of claim 10 further comprising the step of:
Delivering medication or fluids via the central lumen, the at least one non-central lumen, or any independent lumen.

19. The method of claim 10 further comprising the steps:
Separating the multi-lumen as it enters the catheter junction hub into independent lumen that emerge outside of the catheter junction hub;
Sealing the independent lumen with a clamping device.
